(19) United States
(12) Patent Application Publication
(10) Pub. No.: US 2008/0086135 A1
(43) Pub. Date: Apr. 10, 2008

(54) Anterior Spinal Plate

(76) Inventor: Joseph B. White, McLean, VA (US)

Correspondence Address: FISH & RICHARDSON P.C., P.O. BOX 1022, MINNEAPOLIS, MN, 55440-1022, US

(21) Appl. No.: 11/468,691

(22) Filed: Aug. 30, 2006

(51) Int. Cl. A61F 2/30

(52) U.S. Cl. 606/69

(57) ABSTRACT

An anterior spinal plate includes a plate body having a longitudinal extent to span between vertebral bodies. At least one end portion of the plate body is configured to substantially conform to the anterior surface of a vertebral body, and at least one end portion defines a screw hole. The screw hole is positioned relative to a positioning guide such that in use, with the positioning guide against an end plate of a vertebral body and a screw received through the screw hole engaging the vertebral body at an angle of greater than about 20 degrees along the cephalad/caudal axis, the end portion defining the screw hole extends less than about 7 mm along the cephalad/caudal axis of the vertebral body. A method includes placing the plate body against the anterior surface of the spine with the positioning guide against the end plate of the vertebral body, and advancing a screw through the screw hole with the screw engaging the vertebral body at an angle of greater than about 20 degrees along the cephalad/caudal axis while the end portion defining the screw hole extends less than about 7 mm along the cephalad/caudal axis of the vertebral body.

### TECHNICAL FIELD

This invention relates to anterior spinal plates.

### BACKGROUND

Anterior spinal plates used in spine surgery are generally fixed to the anterior portion of the vertebra using screws. Screws are inserted into the vertebra in a direction generally perpendicular to the longitudinal extent of the plate, with most plates permitting an inclination of the inserted screw of approximately 15 degrees in the caudad or cephalad direction. These screws can be fixed at a desired angle after implantation so that there is no change in the angulation between the plate and the screw, or the screws can have a variable angle which permits the screw angle to change after the surgical procedure is completed.

### SUMMARY

To provide better fixation of an anterior spinal plate to the vertebra, and to provide ease of insertion of the screws into the vertebra, an anterior spinal plate is shaped to conform to the surface of the vertebra, minimizes dissection required to access the vertebra for screw placement, and maximizes screw purchase while minimizing the impact on the plate on normal spine motion. The anterior spinal plate is particular adapted to use in the upper cervical spine, where the anterior vertebral surface has a small radius of curvature at the C₂ and C₃ levels and the anterior vertebral surface and inferior end plate surface meet at an acute angle of about 25 degrees, and in the very low cervical spine and upper thoracic spine, where extensive dissection is normally required to provide access for screw placement.

In one aspect, an anterior spinal plate includes a plate body having a longitudinal extent to span between vertebral bodies. The plate body includes a positioning guide and first and second end portions. At least one of the end portions is configured to substantially conform to the anterior surface of a vertebral body, and at least one of the end portions defines a screw hole. The spinal plate includes a screw for receipt by the screw hole. The screw and screw hole are configured and the screw hole is positioned relative to the positioning guide such that in use, with the positioning guide against an end plate of a selected vertebral body, the screw engages the selected vertebral body at an angle of greater than about 20 degrees along the cephalad/caudal axis while the end portion defining the screw hole extends less than about 7 mm along the cephalad/caudal axis of the selected vertebral body.

Embodiments of this aspect may include one or more of the following features.

The screw and screw hole are configured to lock the screw at a set angle of about 35 or 45 degrees. The positioning guide has a length extending in a direction perpendicular to an anterior surface of the plate body of less than about 3 mm.

According to another aspect, an anterior spinal assembly includes a plate body, a screw, and a cage attached to the plate body. The plate body has an end portion configured to substantially conform to the anterior surface of a vertebral body. The plate body defines a screw hole. The screw and screw hole are configured such that in use the screw engages a selected vertebral body at an angle of greater than about 20 degrees along the cephalad/caudal axis.

Embodiments of this aspect may include one or more of the following features.

The cage is removably attached to the plate body, for example, by a fixation device. The fixation device is configured such that after implantation of the cage and plate body, the plate body is removable from the cage by manipulating the fixation device.

The plate body has a longitudinal extent to span between vertebral bodies to be fused, and the plate body includes a positioning guide. The screw and screw hole are configured and the screw hole is positioned relative to the positioning guide such that in use, with the positioning guide against an end plate of the selected vertebral body and the screw engaging the selected vertebral body at an angle of greater than about 20 degrees along the cephalad/caudal axis, the end portion defining the screw hole extends less than about 7 mm along the cephalad/caudal axis of the selected vertebral body.

According to another aspect, an anterior spinal plate includes a plate body having a longitudinal extent to span between vertebral bodies. The plate body includes a positioning guide and first and second end portions. At least one of the end portions is configured to substantially conform to the anterior surface of a vertebral body, and at least one of the end portions defines a screw hole. The screw hole is positioned relative to the positioning guide such that in use, with the positioning guide against an end plate of a selected vertebral body and a screw received through the screw hole engaging the selected vertebral body at an angle of greater than about 20 degrees along the cephalad/caudal axis, the end portion defining the screw hole extends less than about 7 mm along the cephalad/caudal axis of the selected vertebral body.

According to another aspect, a method includes placing a plate body against the anterior surface of the spine with a positioning guide of the plate body against an end plate of a selected vertebral body. The plate body includes first and second end portions. At least one of the end portions is configured to substantially conform to the anterior surface of a vertebral body, and at least one of the end portions defines a screw hole. The method includes advancing a screw through the screw hole. The screw hole is positioned relative to the positioning guide such that the screw engages the selected vertebral body at an angle of greater than about 20 degrees along the cephalad/caudal axis while the end portion defining the screw hole extends less than about 7 mm along the cephalad/caudal axis of the selected vertebral body.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of the fixation of an anterior spinal plate.

FIGS. 2A-2C are various views of a plate body of the anterior spinal plate of FIG. 1.

FIG. 3 is an illustration of the fixation of an alternative embodiment of an anterior spinal plate.

FIG. 4 is an illustration of the fixation of another alternative embodiment of an anterior spinal plate.

FIGS. 5 and 6 are directed to an embodiment in which an anterior spinal plate is removably attached to an interbody cage.

### DETAILED DESCRIPTION

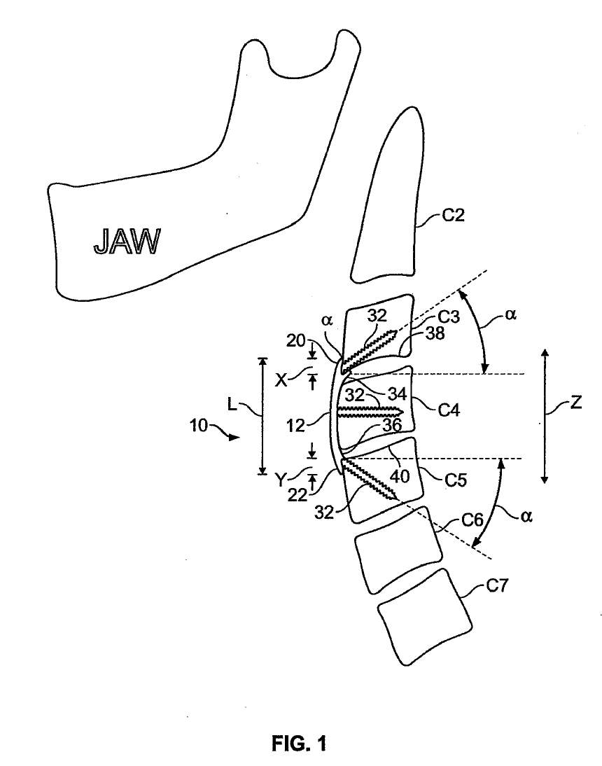

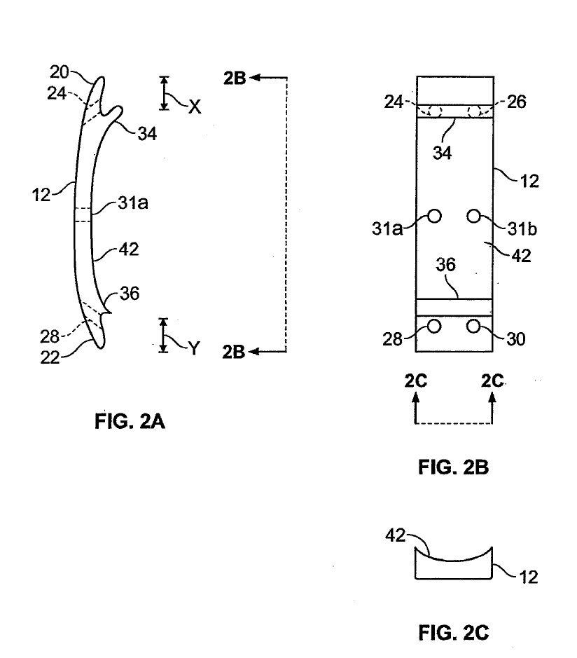

An anterior spinal plate 10, illustrated in FIG. 1, includes a plate body 12 having a longitudinal extent, L, to span between vertebral bodies to be fused, for example, three vertebral bodies at levels C₃ to C₅ of the cervical spine, as illustrated, or at levels C₂ to C₄. In order to achieve better fixation of the anterior plate to the spine, particularly the upper cervical spine, the plate body 12 has first and second end portions 20 and 22 that are contoured to substantially conform to the convex shape of the anterior arch of a respective vertebral body (vertebral bodies C₃ and C₅, as illustrated), as discussed further below with reference to FIGS. 2A-2C.

Each end portion 20, 22 defines a pair of holes 24 and 26, and 28 and 30, respectively (FIG. 2B), each for receiving a screw 32 such that in use the screws 32 engage the vertebral bodies C₃, C₅ at an angle α of greater than about 20 degrees along the cephalad/caudal axis Z (screws 32 engaging vertebral body C₃ being cephlad directed, and screws 32 engaging vertebral body C₅ being caudad directed). The angle α can be a single set angle or a variable angle greater than about 20 degrees that can be fixed at a desired set angle. The plate body 12 further defines a pair of centrally located holes 31a, 31b (FIG. 2B) that receive screws 32 at an angle generally perpendicular to the plate body 12 for engaging center vertebral body 16. The implanted central screws can have a variable angle of about 7 degrees cephlad to 7 degrees caudad, or the central screws can be of a fixed angle variety, which meet the plate at ninety degrees, perpendicular to the plate.

The plate body 12 includes a positioning guide 34 at end portion 20, and a positioning guide 36 at end portion 22. The screw holes are positioned relative to the positioning guides such that in use, with the positioning guides 34, 36 against a respective end plate 38, 40 of the vertebral bodies C₃, C₅ (after removal of the discs), the screw 32 engages the vertebral body at an angle of greater than about 20 degrees along the cephalad/caudal axis while the end portion 20 extends a distance, X, less than about 7 mm, preferably about 6 mm, along the cephalad/caudal axis of the vertebral body C₃ from the end plate 38, and the end portion 22 extends a distance, Y, less than about 7 mm along the cephalad/caudal axis of the vertebral body C₅ from the end plate 40.

The screw angle and the limited extension of the end portion 20 from the guide 34 combine to maximizes screw purchase within the uppermost vertebra to be fused while minimizing the impact of the plate on movement of the vertebra. The screw angle is particularly important in maximizing screw purchase at levels C₂, C₃ and C₄. The angle γ between the anterior vertebral surface and the inferior end plate at levels C₂ and C₃ is typically about 20 to 25 degrees. To place a screw perpendicularly through a plate into a vertebral body having this angle would require that the plate and screw be placed well above the inferior surface to keep the screw within the vertebra. This placement of the plate introduces the risk of interfering with the normal motion segment which lies above the vertebra which has been fused. This need to place the plate higher on the vertebral body and its associated risk can be avoided by angling the screw in the cephalad direction by an amount greater than 20 degrees. In particular, an angle α of 35 to 40 degrees cephlad permits the plate to remain on the lower half of the vertebral body to which it has been affixed. The placement of the guide 34 determines the extent to which the end portion 20 of the plate extends along the anterior surface of the vertebra.

The screw angle at both the uppermost vertebra and the lowest vertebra provide ease of access for insertion of the screws while minimizing the required dissection. In systems in which the screw must approach the plate in an almost perpendicular fashion, it is necessary to do significant soft tissue dissection in order to make a perpendicular approach to the plate with the screw. A more cephalad-oriented screw placed into the highest vertebra obviates the need for a significant amount of soft tissue dissection perpendicular to the orientation of the plate. A more caudal-oriented screw placed into the lowest vertebra has a similar advantage, particularly when affixing a plate to the lower cervical spine or the upper thoracic region. There are situations in which a corpectomy or discectomy needs to be carried out at the C₆-C₇ area or C₇-T₁ area. In these circumstances, it is occasionally necessary to divide the sternum in order to gain access to this area. A screw which is directly caudally into either the T₁ vertebra or T₂ vertebra, which is placed with a 30 to 45 degree caudad orientation, can make it possible to instrument that region without carrying out a sternal splitting approach.

The configuration of the screw and/or the screw hole can be such that the screw is a fixed angle screw that can be positioned at a single angle (for example, 45 degrees), or the screw is a variable-angle screw permitting about 12 degrees of angulation about the insertion angle. Various designs for fixed angle screws and variable-angle screws are known in the art.

Referring to FIGS. 2A-2C, the positioning guides are of limited length, with the positioning guide 34 extending less than about 3 mm from an interior surface 42 of the plate 12, and positioning guide 36 extending less about 3 mm from the interior surface 42. For example, the length of the positioning guides 34 and 36 is selected such that the guides do not extend into the area where fusion is to take place. Surface 42 is contoured both in the cephalad/caudad direction and in the lateral direction to conform to the shape of the surface of the vertebra thus aligning the plate to the anterior vertebral body. In the lateral direction, the surface 42 is concave where the plate makes contact with the anterior cervical body, thus facilitating contact between the plate and the vertebral body at the position where the screw enters the vertebral body. This anatomic radius of curvature is particularly applicable at the C₂ and C₃ levels, which have a radius of approximately 1 cm.

Plate 12 can have a length that spans more than three vertebral bodies.

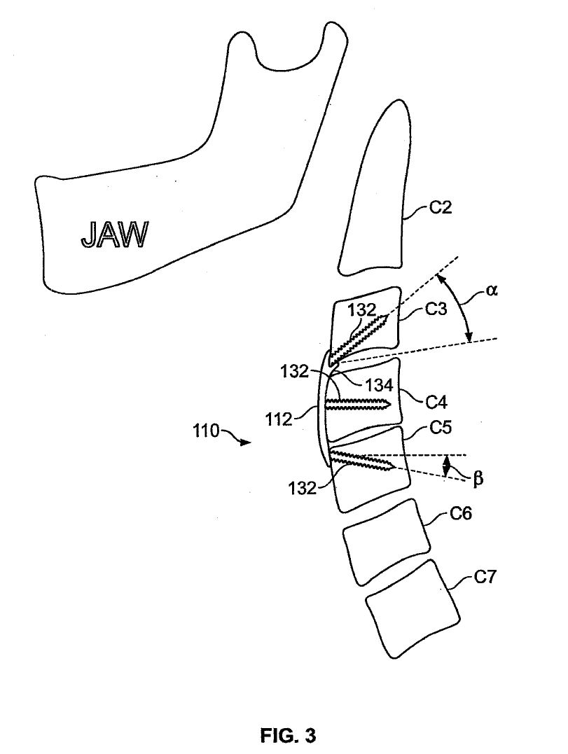

Other embodiments are within the scope of the claims. For example, referring to FIG. 3, an anterior spinal plate 110 includes a plate body 112 that corresponds to plate body 12 of FIG. 1 except that screws 132 engage the lowermost vertebral body C₄ or C₅ at an angle β less than angle α, that is, at an angle β less than 20 degrees along the cephalad/caudal axis Z, for example, at a zero degree angle in which the screw is perpendicular to the plate. The screw engaging the lowermost vertebral body can be of the fixed or variable-angle variety. The plate body 112 includes a positioning guide 134 corresponding to positioning guide 34 of FIG. 1, but does not include a positioning guide corresponding to positioning guide 36 of FIG. 1. The plate configuration of FIG. 3 is particularly suitable for instrumentation of C₂, C₃, or C₄ to a variable number of vertebrae below it.

Plate 110 can also be used in situations where a plate spanning only two vertebral bodies is desired, as discussed below, with the upper screw at angle α being inserted at C₃ and the lower screw at angle β being inserted at C₄.

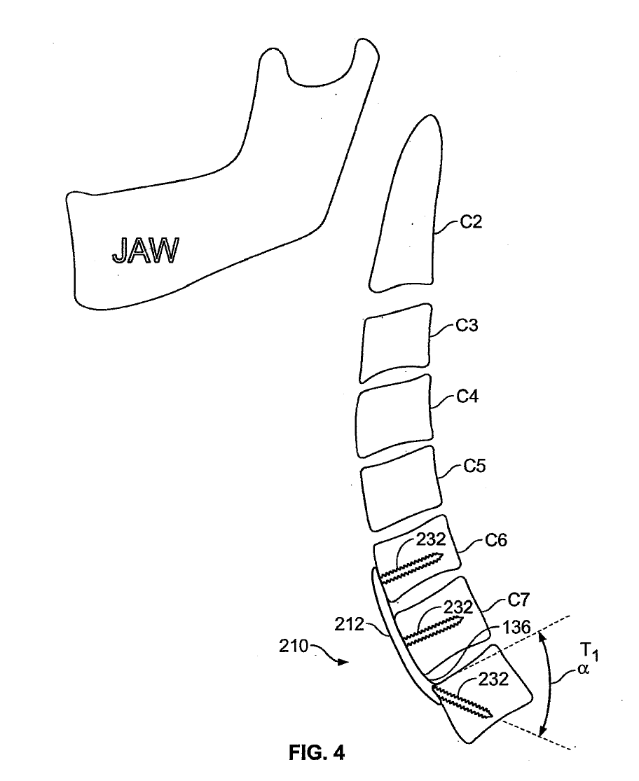

The anterior spinal plate 210 of FIG. 4 is particularly adapted for use in fusing vertebra at the C₆, C₇, to T₁ levels, with the screws at the lowers level being at about a 45 degree caudad angle. Anterior spinal plate 210 includes a plate body 212 that corresponds to plate body 12 of FIG. 1 except that screws 232 engage the vertebral body C₃ at an angle generally perpendicular to the plate body 212, using either a fixed or variable-angle screw. The plate body 212 includes a positioning guide 136 corresponding to positioning guide 36 of FIG. 1, but does not include a positioning guide corresponding to positioning guide 34 of FIG. 1. The length of this plate can vary to fuse, for example, up to five vertebrae, for example, C₄ to T₁. The radius of curvature of this plate in the lateral direction is generally less than the radius of curvature for plates being affixed to the upper cervical spine at C₂ and C₃.

For use in a corpectomy of C₄ and C₅, the plate 10 of FIG. 1 can be sized to span from C₃ to C₆, with cephalad and caudal angles at C₃ and C₆ of about 45 degrees. In the case of a multilevel corpectomy procedure, this plate construct can be used in conjunction with an interbody cage.

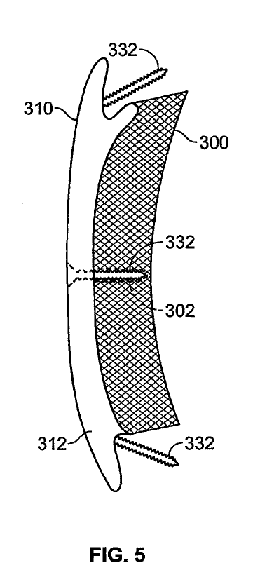

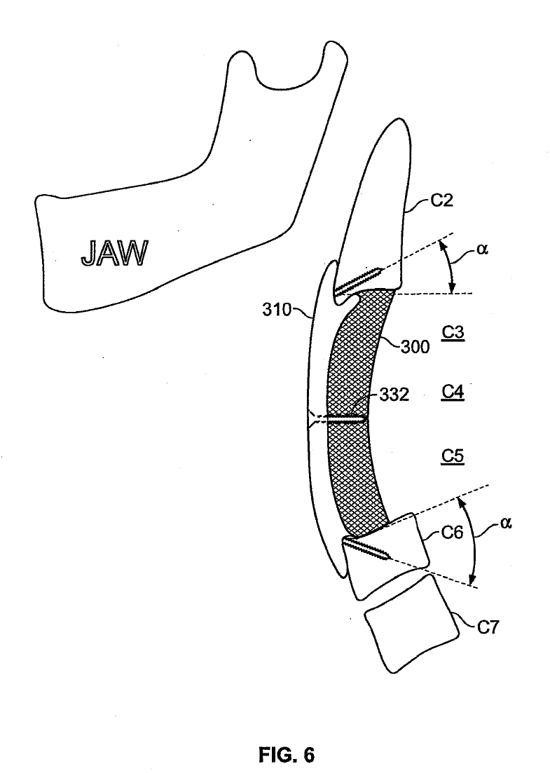

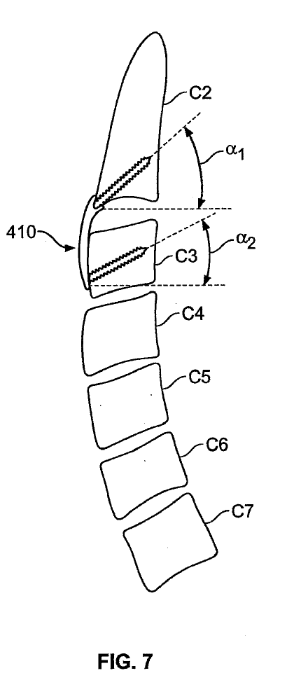

Referring to FIGS. 5 and 6, when one or more vertebral bodies are to be removed in a corpectomy procedure and replaced by a cage 300, an anterior spinal plate 310 including a plate body 312 corresponding to plate body 12 of FIG. 1 removably attached to the cage using central screws 332 can be used to position the cage and maintain the cage position. Cage 300 defines threaded screw holes 302 for receiving central screws 332. As illustrated in FIG. 6, three vertebral bodies at the C₃-C₅ levels have been removed and the cage 300 inserted in their place. The attachment of the plate body 312 to the cage 300 as well as to vertebral bodies C₂ and C₆, maintains the position of the cage 300 during bone ingrowth into the cage, and the attachment of the plate body 312 to the cage 300 creates a solid, one-piece construct that limits the possibility that the interbody cage would be dislodged posteriorly into the spinal canal. After implantation of the cage 300 and the plate body 312, the plate body is removable from the cage by loosening central screws 332. The plate body 312 can be completely removed from the patient by loosening the remaining screws 332. Since the plate is removably attached to the interbody device, after a solid arthrodesis has been achieved, the plate can be removed without disturbing the interbody fusion.

The anterior spinal plate can have a length that only spans two vertebral bodies. Referring to FIG. 7, an anterior spinal plate 410 that spans only two vertebral bodies is particularly adapted for use in treatment of an unstable hangman's fracture in an individual who cannot tolerate a halo vest. In this situation, a C₂-C₃ anterior plate provides stability and obviates the need for a halo vest. This plate construct can also be used to treat a significantly unstable ligamentous injury between C2 and C3. At the upper vertebra, the angle α₁ is preferably about 45 degrees and the plate curvature conforms to the anterior surface of the vertebra. At the lower vertebra, the angle α₂ is preferably about 55 to 60 degrees and the plate curvature conforms to the anterior surface of the vertebra.

A number of embodiments of the invention have been described. Nevertheless, it will be understood that various modifications may be made without departing from the spirit and scope of the invention. For example, one screw along a central, longitudinal axis of the plate can replace the pairs of screws at the lower, center, and/or upper part of the plate. The plate can have a variable length, and can be bendable along the length of the plate. Accordingly, other embodiments are within the scope of the following claims.

What is claimed is:

1. An anterior spinal plate, comprising:
a plate body having a longitudinal extent to span between vertebral bodies, the plate body including a positioning guide and first and second end portions, at least one of the end portions configured to substantially conform to the anterior surface of a vertebral body, at least one of the end portions defining a screw hole; and
a screw for receipt by the screw hole, the screw and screw hole being configured and the screw hole being positioned relative to the positioning guide such that in use, with the positioning guide against an end plate of a selected vertebral body, the screw engages the selected vertebral body at an angle of greater than about 20 degrees along the cephalad/caudal axis while the end portion defining the screw hole extends less than about 7 mm along the cephalad/caudal axis of the selected vertebral body.

2. The anterior spinal plate of claim 1 wherein the screw and screw hole are configured to lock the screw at a set angle.

3. The anterior spinal plate of claim 2 wherein the angle is about 35 degrees.

4. The anterior spinal plate of claim 2 wherein the angle is about 45 degrees.

5. The anterior spinal plate of claim 1 wherein the positioning guide has a length extending in a direction perpendicular to an anterior surface of the plate body of less than about 3 mm.

6. An anterior spinal assembly, comprising:
a plate body including an end portion configured to substantially conform to the anterior surface of a vertebral body, the plate body defining a screw hole;
a screw for receipt by the screw hole, the screw and screw hole being configured such that in use the screw engages a selected vertebral body at an angle of greater than about 20 degrees along the cephalad/caudal axis; and
a cage attached to the plate body.

7. The anterior spinal assembly of claim 6 wherein the screw and screw hole are configured to lock the screw at a set angle.

8. The anterior spinal plate of claim 7 wherein the angle is about 35 degrees.

9. The anterior spinal plate of claim 7 wherein the angle is about 45 degrees.

10. The anterior spinal plate of claim 6 wherein the cage is removably attached to the plate body.

11. The anterior spinal plate of claim 10 wherein the cage is removably attached to the plate body by a fixation device, the fixation device being configured such that after implantation of the cage and plate body, the plate body is removable from the cage by manipulating the fixation device.

12. The anterior spinal plate of claim 6 wherein the plate body has a longitudinal extent to span between vertebral bodies to be fused, and the plate body includes a positioning guide.

13. The anterior spinal plate of claim 12 wherein the screw and screw hole are configured and the screw hole is positioned relative to the positioning guide such that in use, with the positioning guide against an end plate of the selected vertebral body and the screw engaging the vertebral body at an angle of greater than about 20 degrees along the cephalad/caudal axis, the end portion defining the screw hole extends less than about 7 mm along the cephalad/caudal axis of the selected vertebral body.

14. An anterior spinal plate, comprising:
a plate body having a longitudinal extent to span between vertebral bodies, the plate body including a positioning guide and first and second end portions, at least one of the end portions configured to substantially conform to the anterior surface of a vertebral body, at least one of the end portions defining a screw hole, the screw hole being positioned relative to the positioning guide such that in use, with the positioning guide against an end plate of a selected vertebral body and a screw received through the screw hole engaging the selected vertebral body at an angle of greater than about 20 degrees along the cephalad/caudal axis, the end portion defining the screw hole extends less than about 7 mm along the cephalad/caudal axis of the selected vertebral body.

15. A method, comprising:
placing a plate body against the anterior surface of the spine with a positioning guide of the plate body against an end plate of a selected vertebral body, the plate body including first and second end portions, at least one of the end portions configured to substantially conform to the anterior surface of a vertebral body, at least one of the end portions defining a screw hole; and
advancing a screw through the screw hole, the screw hole being positioned relative to the positioning guide such that the screw engages the selected vertebral body at an angle of greater than about 20 degrees along the cephalad/caudal axis while the end portion defining the screw hole extends less than about 7 mm along the cephalad/caudal axis of the selected vertebral body.
